(19) United States
(12) Patent Application Publication
(10) Pub. No.: US 2013/0309212 A1
(43) Pub. Date: Nov. 21, 2013

(54) LACTOBACILLUS SALIVARIUS AND METHOD FOR PREPARING METABOLITE THEREOF, COMPOSITION OF LACTOBACILLUS SALIVARIUS AND METABOLITE THEREOF AND USE OF THE COMPOSITION

(71) Applicants: Ming Zhang, Beijing (CN); Fang Wang, Beijing (CN); Huiyuan Guo, Beijing (CN); Jun Zhu, Beijing (CN); Lu Jiang, Beijing (CN)

(72) Inventors: Ming Zhang, Beijing (CN); Fang Wang, Beijing (CN); Huiyuan Guo, Beijing (CN); Jun Zhu, Beijing (CN); Lu Jiang, Beijing (CN)

(73) Assignee: Fazheng REN, Beijing (CN)

(21) Appl. No.: 13/982,965

(22) PCT Filed: Jan. 17, 2012
(86) PCT No.: PCT/CN2012/070474

§ 371 Date: Aug. 13, 2013

(30) Foreign Application Priority Data
Feb. 1, 2011 (CN) 201110034420.8

(51) Int. Cl. A61K 35/74

(52) U.S. Cl. CPC A61K 35/74
USPC 424/93.45; 435/252.9; 424/780

(57) ABSTRACT

Provided is a Lactobacillus salivarius belonging to Lactobacillus salivarius subsp. Salivarius and deposited in China General Microbiological Culture Collection Center with an accession number CGMCC No: 3606. Further provided is a method for preparing a metabolite of the Lactobacillus salivarius, wherein the Lactobacillus salivarius is as described above. Further provided is a composition, comprising viable strains of the Lactobacillus salivarius and/or the metabolite of the Lactobacillus salivarius prepared as described above. Further provided is a use of the composition in preparation of drugs for prevention and/or treatment of cancers and a method for treating and/or preventing cancers. The viable strains of Lactobacillus salivarius and the metabolite of Lactobacillus salivarius provided in the present invention can both remove the genotoxicity of 4-NQQ in vivo, thus exerting an effect of effectively preventing and/or treating cancers.

### TECHNICAL FIELDS

The present invention relates to a Lactobacillus salivarius, the method for preparing the metabolite thereof, the composition containing the Lactobacillus salivarius or the metabolite thereof, and a method for treating and/or preventing cancers. Particularly, the present invention relates to a Lactobacillus salivarius, a method for preparing the metabolite of Lactobacillus salivarius, a composition and use of this Lactobacillus salivarius.

### BACKGROUND ART

Lactobacillus salivarius is a Gram-positive non-spore bacillus, which is widely distributed in fermentation products containing carbohydrate from plants and animals, and which is also found in warm-blooded animal's mouth, vagina and intestine. It can be isolated from surface of plants, dairy products, meat products, beer, wine, fruit juice, wort, dough, sewage and feces of human and animals. It has strong abilities on degrading carbohydrates but weak abilities on degrading proteins. It widely used in production process of foods such as krauts, pickles, wine and yoghurt.

CN 101538545A disclosed a strain of Lactobacillus salivarius and food composition containing the same. Also disclosed is the fact that this strain can stimulate the immune response and can remove the genotoxicity of 4-nitroquinoline-1-oxide (4-NQO) in vitro with the efficiency of 92.91%. However, the experimental data shows that the above strain has poor ability on removing the genotoxicity of 4-NQO in vivo.

### SUMMARY OF THE INVENTION

The present invention provides a particular strain of Lactobacillus salivarius screened by the inventor, which have strong ability on removing the genotoxicity of 4-NQO in vivo, demonstrated by experimental data.

The present invention provides a strain of Lactobacillus salivarius, belonging to Lactobacillus salivarius subsp. Salivarius, deposited in China General Microbiological Culture Collection Center on Jan. 22, 2010, with an accession number CGMCC No: 3606.

Further provided is the metabolite of the Lactobacillus salivarius as defined above.

Further provided is a method for preparing the metabolite of the Lactobacillus salivarius as defined above, comprising: exposing the viable thalli of the Lactobacillus salivarius with liquid medium under the conditions allowing the Lactobacillus salivarius to survive; obtaining the products of the exposure; and isolating the liquid products from the products of the exposure.

Further provided is a composition, comprising the viable thalli of the Lactobacillus salivarius as defined above and/or the metabolite of Lactobacillus salivarius prepared by the method as defined above as active ingredients.

Further provided is a use of the Lactobacillus salivarius as defined above in preparation of drugs or functional foods for prevention and/or treatment of cancers, wherein the drugs or functional foods comprise the viable thalli of the Lactobacillus salivarius as defined above and/or the metabolite of Lactobacillus salivarius as defined above as active ingredients.

Further provided is a method for treating and/or preventing cancers, comprising preparing a formulation for treating and/or preventing cancers by using the composition as defined above, and administering the formulation to the subject in need.

Lactobacillus salivarius provided in the present invention can be cultured to produce mass of viable thalli, and also can produce metabolite via its metabolism. The viable thalli and metabolite of this Lactobacillus salivarius both can remove the genotoxicity of 4-NQO in vivo. Furthermore, the composition comprising Lactobacillus salivarius and/or the metabolite of Lactobacillus salivarius as defined above can be used to effectively treat and/or prevent cancers, especially for the cancers of the digestive tract.

Other features and advantages of the present invention will be illustrated more thoroughly in the section of detailed description of the invention.

### DETAILED DESCRIPTION OF THE INVENTION

The present invention will be described in details hereafter. It should be understood that the particular embodiments disclosed herein is intended to be illustration and explanation only and should not be construed as any limitation of the invention.

The present invention provides a particular strain of Lactobacillus salivarius screened by the inventor, which have strong ability on removing the genotoxicity of 4-NQO in vivo. This strain of Lactobacillus salivarius belongs to Lactobacillus salivarius subsp. Salivarius, deposited in China General Microbiological Culture Collection Center on Jan. 22, 2010, with an accession number CGMCC No: 3606.

Lactobacillus salivarius provided in the present invention can be cultured to produce mass of viable thalli. The culture conditions have no specific requirements, provided that the Lactobacillus salivarius can proliferate under such conditions. For example, 10⁷ cells can be inoculated on Lactobacillus culture medium and incubated for 8-72 hours under 15-38° C., either in anaerobic or aerobic conditions. Then the culture can be obtained. Said Lactobacillus culture medium can be any suitable Lactobacillus culture medium that is well-known in the art, such as milk and/or Lactobacillus (MRS) medium as described in Yang Jiebin et al., “Lactobacillus-Biological Basis and Applications”, China Light Industry Press, 1996.

The present invention also relates to the isolation of viable thalli of Lactobacillus salivarius from the culture. The isolation methods have no specific requirements, provided that the Lactobacillus salivarius can be enriched from the culture. For example, it can be achieved by centrifugation and/or filtration. The protocols of such centrifugation and/or filtration are well-known in the art and will not be discussed in this document for conciseness.

The present invention further provides the metabolite of the Lactobacillus salivarius as defined above. The term “metabolite” as used herein refers to substances produced by the metabolism of the Lactobacillus salivarius as defined above.

Further provided is a method for preparing the metabolite of the Lactobacillus salivarius, said Lactobacillus salivarius can be Lactobacillus salivarius as defined above, said method may comprising: exposing the viable thalli of the Lactobacillus salivarius with liquid medium under the conditions allowing the Lactobacillus salivarius to survive; obtaining the products of the exposure; and isolating the liquid products from the products of the exposure.

The conditions allowing the Lactobacillus salivarius to survive have no specific requirements, provided that the Lactobacillus salivarius can maintain its living activities, such as the conditions as described in Yang Jiebin et al., “Lactobacillus-Biological Basis and Applications”, China Light Industry Press, 1996.

The ratio of the amount of viable thalli of Lactobacillus salivarius and the liquid medium has no specific requirements and can be common selections used for obtaining the metabolites of viable thalli. Preferable amount of the liquid medium is in the range of 0.5-100 ml, more preferably 1-10 ml, for 10⁶ CFU of the viable thalli of said Lactobacillus salivarius.

The period of the exposure has no specific requirements, provided that the metabolites of Lactobacillus salivarius can disperse in the liquid medium. To obtain more metabolite, the period of the exposure can preferably be 0.3-10 hours, more preferably 1-5 hours.

The selection of the liquid medium has no specific requirements, provided that the Lactobacillus salivarius can maintain its living activities and disperse its metabolites into the liquid medium. To obtain more metabolite, the liquid medium can preferably be one or more selected from the group consisting of water, physiological saline and phosphate buffer saline. More preferably, the liquid medium is water such that the effects of salt ions on bioactivities of the metabolites can be avoided.

The compositions of physiological saline and phosphate buffer saline are well-known in the art and will not be discussed in this document for conciseness.

The liquid products can be used directly as the metabolites of Lactobacillus salivarius, or it can be used as the metabolites of Lactobacillus salivarius after concentration or drying.

The present invention further provides a composition, comprising the viable thalli of the Lactobacillus salivarius as defined above and/or the metabolite of Lactobacillus salivarius prepared by the method as defined above as active ingredients.

The amount of the viable thalli of the Lactobacillus salivarius in said composition can be 10⁵-10¹⁰ CFU/g, preferably 10⁶-10⁹ CFU/g, based on the total weight of the composition.

The amount of dry matter of the metabolite in said composition is 0.1-99.9% by weight, preferably 1-99% by weight, more preferably 10-90% by weight, based on the total weight of the composition.

The term “dry matter” as used herein refers to the residues of the organism after thoroughly drying at a constant temperature of 10-90° C.

The composition which can be used in the invention can include one or more selected from the group consisting of the culture of said Lactobacillus salivarius, such as fermented milk products made by Lactobacillus salivarius fermentation, isolated viable thalli of said Lactobacillus salivarius, the products of exposure of viable thalli of said Lactobacillus salivarius to said liquid medium, the liquid product obtained by removal of solid substances from said products of exposure, and the products obtained after the concentration or drying of said liquid product.

The compositions of the present invention can be functional food compositions, which comprise the viable thalli of said Lactobacillus salivarius and/or the metabolite of Lactobacillus salivarius as defined above as active ingredients, and food. Generally, the amount of the viable thalli of the Lactobacillus salivarius in said composition can be 10⁵-10¹⁰ CFU/g, preferably 10⁶-10⁹ CFU/g, based on the total weight of the composition, and/or, the amount of dry matter of the metabolite in said composition is 0.1-99.9% by weight, preferably 1-99% by weight, more preferably 10-90% by weight, based on the total weight of the composition.

The food contained in the functional food composition of the invention can be any type of food, such as fruit juice products, milk products, bean products, and the like. Said functional food composition may further comprise conventional additives, such as spices, minerals, vitamins, stabilizers, thickeners, preservatives and the like.

The composition of the invention can be a pharmaceutical composition comprising the viable thalli of the Lactobacillus salivarius and/or the metabolite of Lactobacillus salivarius as defined above as active ingredients and a pharmaceutically acceptable adjuvant. The pharmaceutical composition can be in conventional dose forms, such as normal tablets or sugar-coated tablets, pills, troches, capsules, drops, granules, ointments, creams or gels. Suitable types of adjuvant can be selected according to the dose form to be formulated. The dose unit can be adjusted according to the effective amount of the viable thalli of Lactobacillus salivarius and/or the metabolites of Lactobacillus salivarius. The effective amount of viable thalli of Lactobacillus salivarius and/or the metabolites of Lactobacillus salivarius will depend on the age and conditions of the subjects, and the preferable range will be: 10⁵-10¹² CFU/kg body weight per day for the viable thalli of the Lactobacillus salivarius, or 10-10000 mg/kg body weight per day for the dry matter of the metabolites of Lactobacillus salivarius. Generally, the amount of the viable thalli of the Lactobacillus salivarius in said composition can be 10⁵-10¹⁰ CFU/g, preferably 10⁶-10⁹ CFU/g, based on the total weight of the composition; and/or, the amount of dry matter of the metabolite in said composition is 0.1-99.9% by weight, preferably 1-99% by weight, more preferably 10-90% by weight, based on the total weight of the composition.

The composition provided by the invention can be used in preparation of drugs or functional foods for prevention and/or treatment of cancer. The composition provided by the invention can be used in preparation of functional foods for prevention of cancer, or can be used in preparation of drugs for prevention and/or treatment of cancer.

Said drugs or functional foods comprise the viable thalli of Lactobacillus salivarius and/or the metabolite of Lactobacillus salivarius as defined above as active ingredients.

The present invention further provides a method for treating and/or preventing cancers, comprising preparing a formulation for treating and/or preventing cancers by using a composition as defined above, and administering the formulation to the subject in need.

The cancers can be cancers of digestive tract, such as oral cancer, esophageal cancer, gastric cancer and colon cancer.

The drugs of the can be administered orally or rectally.

The volume of liquid or gas described in the invention all refer to the volume under the standard state.

The present invention is further illustrated by the following examples.

### EXAMPLE 1

This example describes the viable thalli of Lactobacillus salivarius of the invention.

Lactobacillus salivarius strain CGMCC 3606 is inoculated on 10 L of Lactobacillus medium (MRS medium, purchased from Hufeng Biotechnology Ltd, Shanghai) by sterilized inoculation ring. The Lactobacillus salivarius is incubated under anaerobic conditions for 12 hours at 37° C. The resulted culture is centrifuged at 4000 g for 10 min. after the supernatant is removed, viable thalli of Lactobacillus salivarius is obtained.

### EXAMPLE 2

This example describes the method for preparing the metabolites of Lactobacillus salivarius of the invention.

10¹³ CFU of viable thalli of Lactobacillus salivarius obtained from Example 1 is resuspended in 2 L deionized water. The suspension is kept at 37° C. for 2 hours and then centrifuged at 4000 g for 10 min. The supernatant is isolated, which can serve as the metabolites of Lactobacillus salivarius (the content of dry matter is 0.1%).

### COMPARATIVE EXAMPLE 1

Another batch of viable thalli of Lactobacillus salivarius is prepared according to the similar procedure of Example 1, except for using the strain of CGMCC 2263 as disclosed in CN 101538545A.

### TESTING EXAMPLE 1

This testing Example tests viable thalli of Lactobacillus salivarius obtained from Example 1 and comparative Example 1 and the metabolites obtained from Example 2 on their abilities of removing the genotoxicity of 4-NQO in vivo.

FD334 rats (180-220 g for body weight, purchased from Vitalriver, Beijing) were divided into six groups (15 animals per group) and feed water formulated according to Table 1 for 7 weeks. 4-NQO was purchased from Sigma. Then, all rats were normally feed for 25 weeks and were observed for incidence of oral cancer according to the method as described in WHO Collaborating Centre for Oral Precancerous Lesions, Definition of leukoplakia and related lesions: An aid to studies on oral precancer. Leukoplakia and related lesions. 46(4): 518-539. 1976. The results were summarized in Table 1.

TABLE 1

| | | | Metabolites | |
|---|---|---|---|---|
| | | Viable thalli of | of | |
| | | Lactobacillus | Lactobacillus | Incidence |
| | 4-NQO | salivarius | salivarius | of oral |
| Group | (ppm) | (Example, CFU/g) | (Volume %) | cancer (%) |
| Control | 20 | 0 | 0 | 65 |
| 1 | 20 | Example 1, 5 × 10⁶ | 0 | 33 |
| 2 | 20 | Example 1, 5 × 10⁸ | 0 | 7 |
| 3 | 20 | Example 1, 5 × 10¹⁰ | 0 | 0 |
| 4 | 20 | Comparative Example | 0 | 58 |
| | | 1, 5 × 10¹⁰ | | |
| 5 | 20 | 0 | 100 | 13 |

It can be seen from the Table 1 that the viable thalli obtained from Example 1 and the metabolites obtained from Example 2, when administered with 4-NQO, could effectively reduce the incidence of oral cancer induced by of 4-NQO. That is, they possess high abilities on removing the genotoxicity of 4-NQO in vivo, which can be used to prevent the oral cancer.

### TESTING EXAMPLE 2

This testing Example tests viable thalli of Lactobacillus salivarius obtained from Example 1 and comparative Example 1 and the metabolites obtained from Example 2 on their abilities of treating the oral cancer induced by 4-NQO.

FD334 rats (180-220 g for body weight, purchased from Vitalriver, Beijing) were divided into six groups (15 animals per group) and feed water containing 20 ppm 4-NQO for 7 weeks. Then, all rats were feed water formulated according to Table 2 for 25 weeks and were observed for incidence of oral cancer according to the method as described in WHO Collaborating Centre for Oral Precancerous Lesions, Definition of leukoplakia and related lesions: An aid to studies on oral precancer. Leukoplakia and related lesions. 46(4): 518-539. 1976. The results were summarized in Table 2.

TABLE 2

| | | Metabolites of | |
|---|---|---|---|
| | Viable thalli of | Lactobacillus | |
| | Lactobacillus salivarius | salivarius | Incidence of oral |
| | (Example, CFU/g) | (Volume %) | cancer (%) |
| Control | 0 | 0 | 67 |
| 1 | Example 1, 5 × 10⁶ | 0 | 27 |
| 2 | Example 1, 5 × 10⁸ | 0 | 20 |
| 3 | Example 1, 5 × 10¹⁰ | 0 | 7 |
| 4 | Comparative Example | 0 | 58 |
| | 1, 5 × 10¹⁰ | | |
| 5 | 0 | 100 | 13 |

It can be seen from Table 2 that the viable thalli obtained from Example 1 and the metabolites obtained from Example 2, when administered to rats treated by 4-NQO, could effectively reduce incidence of oral cancer induced by of 4-NQO. That is, they can be used to treat the oral cancer induced by 4-NQO.

### TESTING EXAMPLE 3

This testing Example tests viable thalli of Lactobacillus salivarius obtained from Example 1 and comparative Example 1 and the metabolites obtained from Example 2 on their abilities of preventing esophageal cancer.

40 New Zealand rabbits suffering from esophageal cancer were prepared according to the literature (L I Ning et al., Establishment of a VX2 Esophageal Carcinoma Model in Rabbits, Chinese Journal of Clinical Oncology, 35(12), 2008) and divided into 4 groups (10 animals per group). Then, all rabbits were feed water formulated according to Table 3 for 32 weeks and were observed for incidence of esophageal cancer according to the method as described in the literature above. The results were summarized in Table 3.

TABLE 3

| | Viable thalli of | Metabolites of | |
|---|---|---|---|
| | Lactobacillus | Lactobacillus | Incidence of |
| | salivarius | salivarius | esophageal cancer |
| | (Example, CFU/g) | (Volume %) | (%) |
| Control | 0 | 0 | 100 |
| 1 | Example 1, 5 × 10¹⁰ | 0 | 30 |
| 2 | Comparative Example | 0 | 90 |
| | 1, 5 × 10¹⁰ | | |
| 3 | 0 | 100 | 40 |

It can be seen from Table 3 that the viable thalli obtained from Example 1 and the metabolites obtained from Example 2 possess abilities on preventing esophageal cancer.

### TESTING EXAMPLE 4

This testing Example tests viable thalli of Lactobacillus salivarius obtained from Example 1 and comparative Example 1 and the metabolites obtained from Example 2 on their abilities of preventing gastric cancer.

40 rats suffering from gastric cancer were prepared according to the literature (X U Jianguo et al., Experimental Observations of Gastric Cancers of Rats and Mice induced by Methylcholanthrene, Journal of Jilin University Medical Edition, Vol. 05, 1992) and divided into 4 groups (10 animals per group). Then, all rats were feed water formulated according to Table 4 for 32 weeks and were observed for incidence of gastric cancer according to the method as described in the literature above. The results were summarized in Table 4.

TABLE 4

| | Viable thalli of | Metabolites of | |
|---|---|---|---|
| | Lactobacillus | Lactobacillus | |
| | salivarius | salivarius | Incidence of gastric |
| | (Example, CFU/g) | (Volume %) | cancer (%) |
| Control | 0 | 0 | 100 |
| 1 | Example 1, 5 × 10¹⁰ | 0 | 30 |
| 2 | Comparative Example | 0 | 90 |
| | 1, 5 × 10¹⁰ | | |
| 3 | 0 | 100 | 40 |

It can be seen from Table 4 that the viable thalli obtained from Example 1 and the metabolites obtained from Example 2 possess abilities on preventing gastric cancer.

### TESTING EXAMPLE 5

This testing Example tests viable thalli of Lactobacillus salivarius obtained from Example 1 and comparative Example 1 and the metabolites obtained from Example 2 on their abilities of preventing colon cancer.

40 rats suffering from colon cancer were prepared according to the literature (NIE Chunsheng et al., Establishment of Colon Carcinoma Model in Rats, Journal of Jilin Military Medical College Fourth Military Medical University, Vol. 01, 2001) and divided into 4 groups (10 animals per group). Then, all rats were feed water formulated according to Table 4 for 32 weeks and were observed for incidence of colon cancer according to the method as described in the literature above. The results were summarized in Table 5.

TABLE 5

| | Viable thalli of | Metabolites of | |
|---|---|---|---|
| | Lactobacillus | Lactobacillus | |
| | salivarius | salivarius | Incidence of bowel |
| | (Example, CFU/g) | (Volume %) | cancer (%) |
| Control | 0 | 0 | 100 |
| 1 | Example 1, 5 × 10¹⁰ | 0 | 30 |
| 2 | Comparative Example | 0 | 90 |
| | 1, 5 × 10¹⁰ | | |
| 3 | 0 | 100 | 40 |

It can be seen from Table 5 that the viable thalli obtained from Example 1 and the metabolites obtained from Example 2 possess abilities on preventing colon cancer.

1. A Lactobacillus salivarius, which is belonging to Lactobacillus salivarius subsp. Salivarius and deposited in China General Microbiological Culture Collection Center with an accession number CGMCC No: 3606.

2. A metabolite of the Lactobacillus salivarius of claim 1, wherein the metabolite is prepared by the method comprising:
exposing the viable thalli of the Lactobacillus salivarius with liquid medium under the conditions allowing the Lactobacillus salivarius to survive;
obtaining the products of the exposure; and
isolating the liquid products from the products of the exposure.

3. (canceled)

4. The metabolite of claim 2, wherein the amount of the liquid medium used is 0.5-100 ml per 10⁶ CFU of the viable thalli of the Lactobacillus salivarius, and the period for the exposure is 0.3-10 hours.

5. The metabolite of claim 2, wherein the liquid medium is one or more selected from the group consisting of water, physiological saline, and phosphate buffer saline.

6-10. (canceled)

11. A method for treating and/or preventing cancers, comprising:
administering to a subject in need thereof, a formulation comprising
a viable thalli of a Lactobacillus salivarius, which is belonging to Lactobacillus salivarius subsp. Salivarius and deposited in China General Microbiological Culture Collection Center with an accession number CGMCC No: 3606,
the metabolite of Lactobacillus salivarius of claim 2, or
a combination thereof.

12. The method of claim 11, wherein the cancer is a cancer of digestive tract.

13. The method of claim 11, wherein the cancer is at least one selected from the group consisting of oral cancer, esophageal cancer, gastric cancer, and colon cancer.

14. The method of claim 11, wherein the viable thalli and/or the metabolite is administered orally or rectally.
